(19) United States
(12) Patent Application Publication
(10) Pub. No.: US 2005/0272662 A1
(43) Pub. Date: Dec. 8, 2005

(54) Self-assembled peptide-amphiphiles & self-assembled peptide nanofiber networks presenting multiple signals

(76) Inventors: Samuel I. Stupp, Chicago, IL (US); Krista L. Niece, Evanston, IL (US); Jeffrey D. Hartgerink, Pearland, TX (US)

Correspondence Address: REINHART BOERNER VAN DEUREN S.C.;ATTN: LINDA GABRIEL, DOCKET COORDINATOR, 1000 NORTH WATER STREET, SUITE 2100, MILWAUKEE, WI, 53202, US

(21) Appl. No.: 10/668,672

(22) Filed: Sep. 23, 2003

Related U.S. Application Data
(60) Provisional application No. 60/413,101, filed on Sep. 23, 2002.

(51) Int. Cl. A61K 38/08; C07K 7/06

(52) U.S. Cl. 514/17; 530/329

(57) ABSTRACT

The present invention provides a mixture of self-assembling peptide-amphiphiles with complementary charges whose design and function is patterned after proteins having biological functions. The oppositely charged peptide amphiphiles may be self-assembled by combining them in a charge equivalent ratio. Variations of structural peptide sequences in the oppositely charged peptide-amphiphiles enable the assembled nanofibers to exhibit two or more biologically relevant signals.

### CROSS-REFERENCES

The present application claims priority to U.S. Provisional Patent Application Ser. No. 60/413,101, filed Sep. 23, 2002, the contents of which are incorporated herein by reference in their entirety.

### GOVERNMENT INTEREST

The U.S. Government may have certain rights to this invention pursuant to Grant from the: (i) U.S. Department of Energy, Grant No. DE-FG02-00ER45810, (ii) Air Force Office of Scientific Research, Grant No. F49620-00-1-0283, and (iii) National Science Foundation, Grant No. DMR-9996253 to Northwestern University.

### BACKGROUND OF THE INVENTION

Techniques of tissue engineering employing biocompatible scaffolds provide viable alternatives to prosthetic materials currently used in prosthetic and reconstructive surgery (e.g. craniomaxillofacial and spinal surgery). These materials also hold promise in the formation of tissue or organ equivalents to replace diseased, defective, or injured tissues. Biocompatible scaffolds can be used to form biodegradable materials which may be used for controlled release of therapeutic materials (e.g. genetic material, cells, hormones, drugs, or pro-drugs) into a predetermined area. Importantly, multiple peptide signals may be used in the same supramolecular structure to accomplish different and potentially synergistic effects over the presentations of a single peptide signal. Most polymers used today to create these scaffolds, such as polylactic acid, polyorthoesters, and polyanhydrides, are difficult to mold and, result in, among other things, poor cell attachment and poor integration into the site where the tissue engineered material is utilized. With some exceptions, they also lack biologically relevant signals. Importantly, multiple peptide signals may be used in the same supramolecular structure to accomplish different and potentially synergistic effects over the presentations of a single peptide signal.

### SUMMARY OF THE INVENTION

Embodiments of the present invention include a peptide-amphiphile composition or its salts comprising a first peptide-amphiphile with a hydrophilic region and an ionic charge, the hydrophilic region having a first biological signal associated with it; a second peptide-amphiphile or addition salt with a hydrophilic region, the hydrophilic region of the second peptide amphiphile having a second biological signal and opposite ionic charge associated with it. The first and second peptides in these peptide-amphiphile composition have oppositely signed charges. The oppositely charged peptide amphiphiles may have the same or different magnitude charge. In these compositions the first peptide and second peptide amphiphile or are mixed/combined in a charge equivalent ratio. Preferably the first peptide or second peptide includes a peptide sequence which promotes adhesion of nerve cells and or those that promote axon outgrowth in cells. For example, the first or second peptide amphiphile may include the amino acid sequences YIGSR or IKVAV. To promote bonding of self assembled peptide amphiphiles, the first or second peptide amphiphile may include an amino acid with a functional moiety capable of intermolecular covalent bond formation.

Another embodiment of the present invention includes compositions comprising self-assembled positively-charged peptide-amphiphiles incorporating a first biological signal and negatively-charged peptide-amphiphiles incorporating a second biological signal. The peptide amphiphiles or their salts in these compositions may include amino acids sequence promoting cell adhesion such as IKVAV and YIGSR.

Another embodiment of the present invention includes compositions comprising an aqueous solution of a first peptide-amphiphile or its salts which has a positive net charge at substantially physiological pH and which includes a first biological signal and an aqueous solution of a second peptide-amphiphile or its salts which has a negative net charge at substantially physiological pH. A method of treating a patient with tissue engineered material comprises administering a peptide-amphiphile composition to a site in need thereof, said peptide-amphiphile composition capable of stimulating or inhibiting a plurality of biological signals at the site and the peptide-amphiphile compositions capable of forming a nanofiber network. The method includes peptide-amphiphile composition that have a first peptide-amphiphile with a first biological signal, having an ionic charge, and a second peptide-amphiphile having an opposite ionic charge. The compositions may be used as a tissue defect filler comprised of a self-assembled peptide-amphiphile compound which itself includes at least two biologically relevant signals.

The present invention provides a system of self-assembling charged peptide-amphiphiles. Preferably, the peptide-amphiphiles' design and function is patterned after naturally occurring proteins. The present invention is generally directed to the utilization of self-assembling molecules, more particularly charged self-assembling. peptide-amphiphiles to form such materials. Even more preferably, the present invention is directed to be sequentially different and oppositely-charged epitopes to be utilized in physiological condition especially with regard to physiological conditions which would benefit from having signals to promote a predetermined physiological condition. There are many applications which would benefit from presentation of multiple signals. One such application is nerve regeneration and spinal cord treatment. Another application is tissue engineered material. In a preferred embodiment of the present invention, self-assembly is utilized to form biocompatible material containing nanofiber networks which have more than one biological signal.

One embodiment of the present invention is a peptide-amphiphile having a charged epitope, preferably along with anpeptide-amphiphile having an oppositely or complimentary charged epitope. In an embodiment of the present invention, the complimentary peptide-amphiphiles induce self-assembly into nanofiber networks.

Another embodiment of the present invention provides a system of self-assembling peptide-amphiphiles with complimentary charged epitopes whose design and function is patterned after proteins having biological signals.

In a preferred embodiment self-assembling peptide-amphiphiles form by combining peptide-amphiphiles with sequentially different and oppositely-charged epitopes at near neutral pH, thus presenting multiple peptide signals in the same supramolecular structure. The respective peptide-amphiphile and the molecular system formed therefrom generally consist of a hydrophobic hydrocarbon tail attached to a relatively hydrophilic peptide sequence. Self-assembly of this peptide-amphiphile (PA) may be induced through pH variation (NH₃, or HCl vapors), positively and negatively charged peptide amphiphiles PA^{+x}, PA^{-Y} where x and y are integers, divalent or polyvalent ion addition, dehydration (drying) or combinations of these among other self assembly inducing conditions. Variations of structural peptide sequences in the PA may enable the assembled nanofibers to be reversibly cross-linked for more or less structural stability, or may allow for control of the rate of self-assembly.

The peptide element of the PAs are preferably carboxyl terminated, so that once assembled into fibers, these fibers may participate in further or carbamide bonding. As shown in FIG. 1, the positively charged peptide-amphiphile is carbamide terminated and the negatively charged peptide-amphiphile may be carboxyl terminated. Of course either or both may be carboxyl terminated.

The versatility and functionality of this self-assembling nanofibrous material may prove to be useful in tissue repair or reconstruction. The term tissue includes muscle, nerve, vascular, and bone tissue and other common understandings of tissue. The present invention may also find application in regulation, inhibition or promotion of axon outgrowth in neurons as well as the regulation, inhibition or promotion of cell-substrate adhesion among nerve cells. The potential for coating these compositions of the present invention on surfaces, such as titanium-based orthopedic implants, may furthermore enhance existing tissue engineering strategies.

### BRIEF DESCRIPTION OF THE FIGURES

Various aspects and applications of the present invention will become apparent to the skilled artisan upon consideration of the brief description of the figures and the detailed description of the invention, which follows:

FIG. 1 illustrates the chemical structure of examples of peptide-amphiphiles having opposite charges and unique biological signal portions;

FIG. 2 is an transmission electron micrograph of nanofibers formed by self assembly of compound 1 and compound 2 in a charge equivalent ratio.

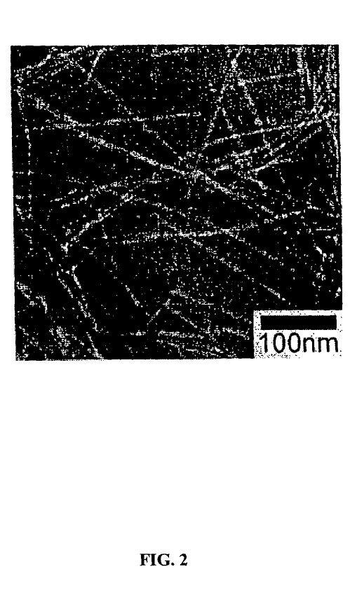

### DETAILED DESCRIPTION OF THE INVENTION

Before the present compositions and methods are described, it is to be understood that this invention is not limited to the particular molecules, compositions, methodologies or protocols described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to a “peptide amphiphile” is a reference to one or more peptide amphiphiles and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of the present invention, the preferred methods, devices, and materials are now described. All publications mentioned herein are incorporated herein by reference. Nothing herein is to be construed as an admission that the invention is not entitled to antedate such disclosure by virtue of prior invention.

The present invention is directed to various modes of self-assembly and controlled self-assembly of charged peptide-amphiphiles. More particularly, preferred embodiments of the present invention are directed to a mixture of two or more charged peptide-amphiphiles which self assemble to form a nanofiber network near physiological conditions. Peptide-amphiphile compositions may include a first peptide-amphiphile having a first biological signal associated therewith and a second peptide-amphiphile having a second biological signal associated herewith. The first and second peptide are oppositely charged; one has a positive ionic charge and the other has a negative ionic charge. The peptide-amphiphile compositions may include amino acids in the peptide sequence which promotes cell-substrate adhesions, a first biological signal, among nerve cells like YIGSR. The peptide-amphiphile composition may include another peptide sequence, a second biological signal, which promotes axon outgrowth in cells like IKVAV. The peptide amphiphiles having the unique biological signal may self assemble to form nanofiber network comprised of a positively-charged peptide-amphiphile incorporating the first biological signal and a negatively-charged peptide-amphiphile incorporating the second biological signal.

The present invention may provide a system of self assembled nanofibers including micells. The self assembled structures are formed from a solution comprising an aqueous solution of a first peptide-amphiphile composition wherein the PA has a positive net charge at substantially physiological pH and which includes a first biological signal and an aqueous solution of a second peptide-amphiphile composition which has a negative net charge at substantially physiological pH and a second biological signal. The solutions may be used sequentially or in combination as a tissue defect filler.

The compositions of the present invention may be used in a method of treating a patient with tissue engineered material comprised of administering a peptide-amphiphile composition to a site in need thereof, the peptide-amphiphile composition capable of stimulating or inhibiting a plurality of biological signals at said site, the peptide-amphiphile compositions capable of forming a nanofiber network. The method includes a peptide-amphiphile composition that is comprised of a first peptide-amphiphile with a first biological signal and having a charge, and a second peptide-amphiphile having a second biological signal and an opposite ionic charge. The compositions may be delivered separately or in combination to a site in need of a tissue engineered material.

Compositions and methods of the present invention include the mixing of two or more peptide amphiphiles (or their addition salts) with biologically relevant signals with opposite charges in charge equivalent ratios to form self-assembled nanofibers or micells, thereby more closely mimicking the body's own extracellular matrix.

Importantly, a combination of a positively and negatively charged amphiphiles allows formation of nanofibers at neutral or physiological pH. Even more importantly, these differently charged amphiphiles contain distinct biological signals.

Table 1 below illustrates representative, non-limiting examples of peptide-amphiphiles with opposite charge and distinct biological signals.

TABLE 1

| | | | | Net |
|---|---|---|---|---|
| | | | | Charge at |
| # | N-terminus | Peptide (N to C) | C-terminus | pH 7 |
| 1 | C16 | AAAAGGGEIKVAV | COOH | −1 |
| 2 | C16 | AAAAGGGKYIGSR | NH₂ | +2 |

The molecules according to the present invention comprise an assembly of three segments: an alkyl tail, a structural peptide, and a functional peptide. These molecules are believed to be conical in shape allowing them to assemble into a cylindrical micelle (a nanofiber) in an aqueous environment with the alkyl tail inside the core of the micelle or nanofiber, and the functional peptide sequence exposed on the surface of the nanofiber.

The alkyl tail has been patterned in large part after the original PA described by Hartgerink, et al, Science, vol 294, pp 1684, (2001) and PNAS vol 99, pp 5133, (2002), the contents of which are incorporated herein by reference in their entirety, where the carbon chain serves as the hydrophobic component of the amphiphile and creates the slender portion of the molecules' conical shape. The structural peptide sequences described herein provide a number of different functions and consist of various amino-acid segments each coupled to the hydrophobic tail. The structural segment in an alternative embodiment includes one or more cysteine amino acids which provides assembled fibers with reversible cross-linking potential. Once assembled into nanofibers, the S—H ligands of the cysteines are believe to be arranged near enough one-another that oxidation of the molecule will enable the formation of stable disulfide bonds. While this cross-link provides structural stability for the molecule, it may be reversed with a reducing agent, such as dithiolthreitol (DTT). The alanine-based structure is not cross-linkable, but avoids the problems of premature molecular crosslinking, which may form between unassembled PA molecules in the presence of oxygen (air). This cysteine-free system may be more appropriate for in situ biological applications where the environment may be more difficult to regulate. The SLSL modification to the system is expected to lead to a slower assembly of the nanofibers. It is believed that the bulky leucine side chains-may require more time to pack into the fiber. A slowed self-assembly may also have greater applications in a functional, in situ environment such as an operating room, where it may be advantageous to have delayed formation of the nano-fibers. The functional hydrophobic head of the peptide is a relatively bulky, charged segment of the molecule, and it serves as the widest region of the conical molecular geometry. Self-assembly of PA mixtures may also allow for the presentation of different amino acid sequences along the length of an assembled fiber.

The peptide-amphiphile compositions of the present invention can be synthesized using preparatory techniques well-known to those skilled in the art—preferably, by standard solid-phase peptide chemistry and addition of an alkyl tail at the N-terminus of the peptide. To induce self-assembly of the charged peptide-amphiphiles, the pH of the solution may be lowered, divalent ions may be added to the solution, and the solution may be subject to dehydration (drying) or other inducing conditions. Preferably self assembly is induced by combining charge equivalent mixtures of positively and negatively charged peptide amphiphiles. According to existing knowledge of amphiphile self-assembly, an alkyl tail with 16 carbon atoms coupled to an ionic peptide should create an amphiphile that assembles in water into cylindrical micelles because of the amphiphiles overall conical shape. The alkyl tails pack in the center of the micelle with the peptide segments exposed to an aqueous environment. These cylindrical micelles can be viewed as fibers in which the chemistry of the peptide region is repetitively displayed on their surface. Similar amphiphile molecules can also be designed to provide micelles having structural shapes that may differ from a fiber like appearance. Other compositions may also be used to induce predetermined geometric orientations of the self-assembled amphiphile peptides.

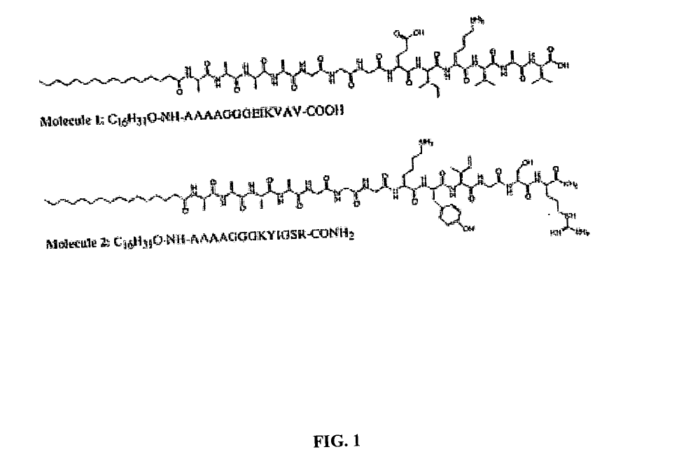

FIG. 1 illustrates the chemical structures of Molecule 1 and Molecule 2 in accordance with a preferred embodiment of the present invention. FIG. 1 also ilustrates the chemical connectivity of a peptide-amphiphile has been described previously indicating three important segments for consideration in the design of the molecule: Segment 1 is generally a simple hydrophobic tail such as an alkyl tail that can be a variety of sizes but should be greater than 6 carbon atoms in length; Segment 2 is a structural segment that includes amino acids that link the alkyl tail to the hydrophilic head group. If cross-linking of peptide amphiphiles or their salts in nanofibers is desired, cysteine amino acids may be utilized in this segment. If cross-linking is not desired, other amino acids such as alanine may be used in this region (e.g. SLSL or AAA as described in more detail herein). The structural segment may also include a flexible linker composed of glycine or other flexible amino acids. In accordance with the present invention, Segment 3 includes the hydrophilic head group and may be comprised of essentially any charged or hydrophilic amino acid such as lysine, arginine, serine, phosphorylated serine, and aspartic acid resulting in a highly charged peptide-amphiphile. As will be discussed further herein, these charged peptide-amphiphiles may be positively or negatively charged and the amino acid sequence similar to biologically relevant signals like IKVAV and YIGSR.

Amino acids useful in the peptide amphiphiles of the present invention include but are not limited to naturally occurring amino acids and artificial amino acids. Incorporation of artificial amino acids such as beta or gamma amino acids and those containing non-natural side chains, and/or other similar monomers such as hydroxyacids are also contemplated, with the effect that the corresponding component is peptide-like in this respect.

The self-assembled peptide-amphiphiles described in this disclosure are modifications of those originally described, by Hartgerink, et al. (See e.g., J. D. Hartgerink, E. Beniash and S. I. Stupp, Science 294, 1683-1688, 2001), which is hereby incorporated in its entirety by reference in its entirety and the synthetic schemes set forth therein apply as well to the present invention. Although the focus of the description is charged PA's or their addition salts presenting mixed biological signals, the present invention is not to be so limited. Various other amphiphile compositions of this invention can be prepared in analogous fashion, as would be known to those skilled in the art and aware thereof, using known procedures and synthetic techniques or straight-forward modifications thereof depending upon a desired amphiphile composition or peptide sequence.

The formation of a self-supporting matrix or solid comprised of these nanofibers under physiological conditions affords the opportunity to utilize this material for a wide range of purposes, e.g., mineralized tissue repair or reconstruction, regulation and inhibition of mineral formation, and coating orthopedic implants or the like.

The present invention provides for a series of peptide-amphiphiles having different sign or opposite charges and peptide sequences mimicking natural peptides. The present invention provides self-assembly at near neutral pH (pH ≈7.4). This permits in vivo injectable applications of the present invention. The charges on the oppositely charged peptide amphiphiles may be the same magnitude (+1, −1) or may differ in magnitude such as (+1, −3) or (+2, −4). Charges on the peptide amphiphiles may be modified by inclusion of amino acids including but not limited to amine, carboxylic acid, or groups like phosphorylated serines.

Different modes of self-assembly of the peptide-amphiphile molecules into cylindrical fibrils and other shapes have been described. This self-assembly generally occurs at predetermined concentrations of peptide amphiphile to form self supporting gel. It has also been found that an addition of polyvalent metal ions may induce gel formation of the negatively charged peptide-amphipfiles at physiological conditions. A number of negatively charged peptide-amphiphiles self-assembled into nanofibers by addition of polyvalent metal ions such as Ca⁺², Mg⁺², Zn⁺², Cd⁺², Fe⁺², Gd⁺³.

In the present invention self-assembly of peptide-amphiphiles may be induced by combining PA's with sequentially different and oppositely-charged epitopes at neutral pH, or near physiological pH, thus presenting multiple peptide signals in the same supramolecular structure. This may have a synergistic effect over the presentation of a single peptide sequence. Preferably the peptide-amphiphile or their addition salts are mixed or combined in a charge equivalent ratio.

Molecule 1 shown in FIG. 1 contains a portion of the laminin amino acid sequence IKVAV, (Ile-Lys-Val-Ala-Val) which is part of the 19-mer peptide (PA222-2), which has been extensively shown to promote axon outgrowth in neurons. Molecule 2 contains the amino acid sequence YIGSR, which has similarly been shown to promote cell-substrate adhesion among nerve cells and also to play a role in axon guidance. The two molecules can be dissolved in pH-adjusted water at a concentration of about 2-30 mg/ml, and preferably about 10 mg/mL. Molecule 1 is completely clear at this concentration; Molecule 2 is translucent. A self-supporting gel forms quickly on mixing the two solutions at neutral pH. Examination of this gel by negative stain TEM reveals cylindrical micelles. Self-assembled peptide amphiphiles of the present invention can include other mixtures of charged peptide amphiphiles.

Biocompatible, biodegradable, gels are useful as a means of delivering templates, which may or may not include isolated cells, into a patient to create an organ equivalent or tissue such as cartilage. The gels promote engraftment and provide three-dimensional templates for new growth. The resulting tissue is generally similar in composition and histology to naturally occurring tissue.

In one embodiment of the present invention, a self-assembling peptide-amphiphile solution is directly injected into a site in a patient, where the self-assembled peptide amphiphile gel organizes into a matrix.

In another embodiment, cells are suspended in a self-assembled peptide-amphiphile gel that is poured or injected into a mold having a desired anatomical shape, then organized to form a matrix which can be implanted into a patient. Ultimately, the self-assembled peptide-amphiphile gel degrades, leaving only the resulting tissue.

In yet another embodiment of the present invention, the peptide-amphiphiles of the present invention are used in conjunction with other tissue engineering material, either as a gel, solid, or liquid and are used to template tissue growth in a pre-determined area on a patient.

Various aspects of the present invention can be described with reference to the peptide-amphiphile as is generally illustrated in FIG. 1, but consistent with broader aspects of this invention. Other compositions can be prepared in accordance with the to invention and used for the self-assembly of micelles.

A peptide-amphiphile mixture makes available a system for the formation of micellular nanofibers in an aqueous environment at neutral and/or physiological pH conditions. Such a combination can be used to assemble nanofibers with a range of residues providing a variety of chemical or biological signals for corresponding cell adhesion, yielding enhanced properties with respect to tissue engineering or regenerative applications. It is contemplated that, alone or in conjunction with the other factors discussed herein, that preferred medical or therapeutic embodiments of such a system can be utilized.

Since in a preferred embodiment of the present invention, the strategy for peptide-amphiphile self-assembly involves mixing two solutions at near physiological pH, and since after mixing the pH remains substantially neutral, it can be expected to have applications in tissue engineering and other medical applications. In particular, this method of forming the peptide-amphiphile nanofibers may be introduced to a patient in a non-invasive fashion by injecting the two liquids which upon mixing form a stable gel presenting both peptide signals.

As stated above, the amphiphile composition(s) of such a system may include a peptide component having residues capable of intermolecular cross-linking. The thiol moieties of cysteine residues can be used for intermolecular disulfide bond formation through introduction of a suitable oxidizing agent or under physiological conditions. Conversely such bonds can be cleaved by a reducing agent introduced into the system or under reducing conditions. The concentration of cysteine residues can also be varied to control the chemical and/or biological stability of the nanofibrous system and therefore control the rate of therapeutic delivery or release of cells or other beneficial agent, using the nanofibers as the carriers. Furthermore, enzymes could be incorporated in the nanofibers to control biodegradation rate through hydrolysis of the disulfide bonds. Such degradation and/or the concentration of the cysteine residues can be utilized in a variety of tissue engineering contexts.

This technology can be used for a variety of purposes. This system of self-assembling nanofibers may have a number of different potential applications in the biomedical and tissue engineering industry. The complimentary nature of the biological portions of the PA provide potentially synergistic applications. For example, the inclusion of both YIGSR and IKVAV provide heretofore unexpected synergistic applications for nerve regeneration.

Aspects of the present invention are illustrated by reference to the following non-limiting examples.

### EXAMPLE 1

Materials and Methods: Abbreviations: PA: peptide-amphiphile, TEM: transmission electron microscopy, DTT: dithiothreitol, EDT: ethanedithiol, TIS: triisopropyl silane, TFA: triflouroacetic acid, HBTU: (2-(1h-benzotriazole-1-yl)-1,1,3,3-tetramethyluronium hexafluorophosphate, DiEA: Diisopropylethylamine; ESI: Electrospray ionization. Except as noted below, all chemicals were purchased from Fisher or Aldrich and used as provided. Amino acid derivatives were purchased from Applied BioSystems and NovaBiochem; derivatized resins and HBTU were also purchased from NovaBiochem. All water used was deionized with a Millipore Milli-Q water purifier operating at a resistance of 18 MW.

The peptide-amphiphiles were prepared on a 0.25 mmole scale using standard FMOC chemistry on an Applied Biosystems 733 A automated peptide synthesizer. Molecule 1 has a C-terminal carboxylic acid and was made using pre-derivatized Wang resin. Molecule 2 has a C-terminal amide and was made using Rink amide MBHA resin. After the peptide portion of the molecules was prepared, the resin was removed from the automated synthesizer and the N-terminus capped with a fatty acid containing 16 carbon atoms. The alkylation reaction was accomplished using 2 equivalents of the fatty acid, 2 equivalents HBTU and 6 equivalents of DiEA in DMF. The reaction was allowed to proceed for at least six hours after which the reaction was monitored by ninhydrin. The alkylation reaction was repeated until the ninhydrin test was negative. Only two couplings were required in each case.

Cleavage and deprotection of the molecules was accomplished with a mixture of TFA and TIS in a ratio of 95:5 for three hours at room temperature. The cleavage mixture and two subsequent TFA washings were filtered into a round bottom flask. The solution was roto-evaporated to a thick viscous solution. This solution was triturated with cold diethylether. The white precipitate was collected by filtration, washed with copious cold ether and dried under vacuum. The molecules were then dissolved in water at a concentration of 10 mg/mL, adjusting the pH to improve solubility. The solution was initially acidic in both cases. In the case of molecule 1, the pH was raised to about pH 8 with 2M and 100 mM KOH, then back-titrated to pH 7. In the case of molecule 2, the molecule was most soluble at low pH, but remained in solution when the pH was raised to 7 using KOH. The molecules were characterized by ESI MS and were found to have the expected molecular weight.

The two peptide amphiphiles were self-assembled into nanofibers by combining 2 parts of Molecule 1 to 1 part of Molecule 2. The molecules also self-assemble independently by the pH mechanism described in a previously.

Samples of the peptide-amphiphiles were prepared for TEM analysis as follows. A small sample of the gel, prepared in bulk as described above, was smeared onto a holey carbon coated TEM grid (Quantifoil). Negative staining with PTA (phosphotungstic acid) was used in this study. In all cases electron microscopy was performed at an accelerating voltage of 200 kV.

All of the embodiments disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention.

While the principles of this invention have been described in connection with specific embodiments, it should be understood clearly that these descriptions are added only by way of example and are not intended to limit, in any way, the scope of this invention. For instance, various peptide-amphiphiles have been described in conjunction with specific residues and corresponding cell adhesion, but other residues can be used herewith to promote a particular cell adhesion and tissue growth on the nanostructures prepared therefrom. Likewise, while the present invention has been described as applicable to biometric material or tissue engineering, it is also contemplated that gels or related systems of such peptide-amphiphiles can be used as a delivery platform or carrier for drugs, cells or other cellular or therapeutic material incorporated therein. Other advantages and features will become apparent from the claims filed hereafter, with the scope of such claims to be determined by their reasonable equivalents, as would be understood by those skilled in the art.

1. A peptide-amphiphile composition comprising:
a first peptide-amphiphile or salt thereof with a hydrophilic region, said region having a first biological signal and an ionic charge associated therewith; and
a second peptide-amphiphile or salt thereof with a hydrophilic region, said region having a second biological signal and an opposite signed ionic charge associated herewith.

2. The peptide-amphiphile compositions of claim 1, wherein the first peptide and second peptide are in a charge equivalent ratio.

3. The peptide-amphiphile composition of claim 1, wherein the first and second peptide-amphiphiles are oppositely charged.

4. The peptide-amphiphile composition of claim 1, wherein said first peptide or said second peptide includes an amino acid sequence which promotes adhesion of nerve cells with said first or second peptide-amphiphiles.

5. The peptide-amphiphile composition of claim 1, wherein said first or second peptide-amphiphile includes the amino acid YIGSR.

6. The peptide-amphiphile composition of claim 1, wherein said first or said second peptide includes a peptide sequence that promotes axon outgrowth in cells.

7. The composition of claim 1, wherein said first or second peptide-amphiphile includes the amino acid sequence IKVAV.

8. The composition of claim 1, wherein the first or second peptide-amphiphile includes an amino acid with a functional moiety capable of intermolecular covalent bond formation.

9. A composition comprising self-assembled positively-charged peptide-amphiphiles incorporating a first biological signal and a negatively-charged peptide-amphiphiles incorporating a second biological signal.

10. The compositions of claim 9 including peptide-amphiphiles with amino acids sequence promoting cell adhesion.

11. The composition of claim 9, wherein said peptide-amphiphiles include amino acid sequences chosen from the group consisting of IKVAV and YIGSR.

12. A composition comprising:
an aqueous solution of a first peptide-amphiphile composition which has a positive net charge at substantially physiological pH and which includes a first biological signal; and
an aqueous solution of a second peptide-amphiphile composition which has a negative net charge at substantially physiological pH.

13. A method of treating a patient with tissue engineered material comprising:
administering a peptide-amphiphile composition to a site in need thereof, said peptide-amphiphile composition capable of stimulating or inhibiting a plurality of biological signals at said site, said peptide-amphiphile compositions capable of forming a nanofiber network.

14. The method of claim 13, wherein said peptide-amphiphile composition is comprised of a first peptide-amphiphile with a first biological signal, having a charge, and a second peptide-amphiphile having an opposite charge.

15. The method of claim 14, wherein said second peptide-amphiphile includes a second biological signal.

16. A tissue defect filler comprised of a self-assembled peptide-amphiphile compound which itself includes at least two biologically relevant signals.


SEQUENCE LISTING (2 sequences)

SEQ ID NO: 1 - protein, 13 aa, artificial sequence
  synthetic peptide
AAAAGGGEIKVAV

SEQ ID NO: 2 - protein, 13 aa, artificial sequence
  synthetic peptide
AAAAGGGKYIGSR